(19) United States
(12) Patent Application Publication
(10) Pub. No.: US 2024/0416041 A1
(43) Pub. Date: Dec. 19, 2024

(54) AUTOINJECTION DEVICE

(71) Applicant: Congruence Medical Solutions, LLC, Baltimore, MD (US)

(72) Inventors: Gautam Nithyanand Shetty, Hanover, MD (US); Lou Castagna, Middletown, PA (US)

(21) Appl. No.: 18/699,918

(22) PCT Filed: Oct. 11, 2022
(86) PCT No.: PCT/US2022/046335

§ 371 Date: Apr. 10, 2024

Related U.S. Application Data
(60) Provisional application No. 63/254,291, filed on Oct. 11, 2021.

(51) Int. Cl. A61M 5/20; A61M 5/32

(52) U.S. Cl. CPC A61M 5/2046; A61M 5/3243; A61M 5/3286

(57) ABSTRACT

Autoinjector including a compressed gas source including a rigid container having a non-rigid sealing structure, and a syringe with a plunger stopper and a seal defining an actuation space between the plunger stopper and the seal. The autoinjector further includes a puncturing needle fluidly coupled to or coupleable to the actuation space. The puncturing needle further being axially aligned to selectively penetrate the non-rigid sealing structure of the compressed gas source upon relative axial movement therebetween to fluidly couple the puncturing needle with the compressed gas source.

This patent disclosure claims priority to U.S. Provisional Application 63/254,291 filed Oct. 11, 2021, which is included herein for all purposes.

### FIELD OF INVENTION

This disclosure generally relates to injectable drug delivery devices, related methods and manufacture. More specifically, this disclosure relates to a drug delivery device that uses an energy source self-contained within the device to administer an injectable therapy.

### BACKGROUND

Self-administration devices are designed to enable patients to administer injectable treatments in a non-clinical setting (for e.g., at home) or typically other non-clinical settings. Conventional syringes require a user to provide the force necessary to administer the injectable drug. This force is characterized using the Hagen Poiseuille equation. In order to aid the user, self-administration devices include a stored energy source such as compressed springs to provide the force necessary to inject the drug. This category of drug delivery devices includes autoinjectors and wearable body injectors (patch pumps).

Autoinjectors were first introduced in the 1970s to help protect soldiers in the event of chemical warfare. Since then, a number of drugs are increasingly injected by patients themselves using an autoinjector. Most commonly used autoinjectors include a compression spring providing the power necessary to administer the injectable drug. Less common are autoinjectors using an electromechanical source of power to drive the injection of the drug.

As progress is being made with development of newer pharmaceutical treatments, there are increasing demands on performance of autoinjectors. In addition, as treatments shift from hospitals and clinics to a home setting, more people will use autoinjectors to treat health conditions, which creates additional demand on autoinjectors to ensure they can be used in an error-free manner.

One of the trends in pharmaceutical development is that increasingly, drug formulations are getting more viscous due to, for example, higher strength (concentration) biologics, larger injection volumes, and long-acting formulations. Higher drug formulation viscosity requires higher injection force or involve longer injection times. In most conventional, spring-based autoinjectors, a stronger spring would need to be employed, which in turn would cause the autoinjector size to increase. Larger autoinjectors are inherently not discreet, which is seen as a key requirement for self-administration. In addition, larger autoinjectors can cause instability during the injection procedure. Spring-based autoinjectors have been reported to have breakage of drug filled glass syringes. Impact shock to glass syringes from springs upon their release would increase in amplitude with stronger springs. Hence, the path to incorporating stronger springs for viscous formulations may not be the most optimal one.

Electromechanical power based autoinjectors are better positioned to provide a more compact device, while providing additional force for viscous formulations. These autoinjectors can be expensive, however. As a result, they are more practical as reusable autoinjectors because of costs and disposal issues.

More recently, compressed gas has been used as a power source in an autoinjector involving miniaturized compressed gas cylinders. Compressed gas autoinjectors present a number of design challenges. The compressed gas cylinder is typically made of a metal with a welded seal. Breaking the welded seal releases the gas, which in turn is directed to advance a plunger rod sliding in a hermetically sealed cylinder; the plunger rod in turn pushes the plunger stopper to inject the drug. Most single-syringe autoinjectors include this plunger rod.

Since release of the compressed gas involves breaking a welded seal, high actuation forces are required. Hence, compressed gas autoinjectors typically require levered actuator to make it practical for use. Extensive plumbing is involved to transport the compressed gas to the plunger stopper.

It is also important to ensure that the puncturing pin used to break the welded seal is hermetically sealed and also the transport of compressed gas to the syringe occurs with no leaks. Leakage of compressed gas during storage and during the injection step are primary complaints of compressed gas powered autoinjectors. The compressed gas is typically an inert gas such as nitrogen, carbon dioxide or argon. The release of compressed gas can also cause recoil, which may result in accidental removal of the injection needle from the injection site.

When a plunger rod advanced by compressed gas is employed, the applied force on the plunger stopper can be large. If this applied force is not coaxial, the plunger rod can blow by the plunger stopper resulting in an error in drug delivery and breach of container closure integrity.

Maintenance of flow rate can be achieved by either ensuring that the drug volume is much lower than total volume that the gas from the compressed gas source can occupy after it is punctured. Another approach is to incorporate a dual phase gas in compressed gas chamber.

Despite the foregoing, compressed gas as power source has significant advantages. The power source is compact. Also, as the injection volume increases and syringe cross-section increases, the available force to drive the plunger stopper in the syringe using a compressed gas source increases for the same pressure. An autoinjector with a compressed gas power source is more practical to be disposable, which may be a benefit with certain drugs.

New users of autoinjectors inherently have difficulty using autoinjectors in an error free manner. One study showed that 69% of study participants prematurely removed autoinjector from the injection site before the injection was complete when operating with no instructions. This can be particularly problematic for infrequently injected drugs; patients in this case may not have access to a replacement. This lost dose is driven by the fact that needle-safety mechanism is actuated immediately after the autoinjectors is removed from the injection site. The drug is expelled out of the autoinjector even though the needle safety shield is locked. This locking occurs irrespective of whether the complete dose is administered or not. Addressing this technology gap can alleviate a significant therapy compliance burden.

### SUMMARY OF THE INVENTION

The disclosed invention details a compressed gas source incorporated into multiple embodiments of an autoinjector. Embodiments disclosed herein aim to improve upon shortcomings of other compressed gas autoinjector technologies and other autoinjectors. Embodiments of the autoinjector include features to improve usability and address some of the technology gaps in current autoinjectors. Novel features disclosed herein could be applied to autoinjectors not having a compressed gas power source.

Disclosed invention also outlines methods of manufacturing.

The disclosed invention includes a compressed gas source consisting of a container having a closure element that pierceable, yet hermetically seals around the piercing element (such as a sharp, hollow metal tube/needle). When this tube is removed, the pierceable closure element seals again maintaining high pressure within the compressed gas source.

This unique ability enables several improvements over the prior art.

It is envisioned that utility of this compressed gas source beyond autoinjectors includes, but not limited to, wearable body injectors, drug transfer devices.

The disclosed invention is a compact, high performance autoinjector powered by a compressed gas source.

According to an aspect of this disclosure, the is provided an autoinjector for use in the injection of an injectable drug with assistance of a compressed gas. The autoinjector includes a compressed gas source and a syringe mounted together by a housing. The compressed gas source includes a rigid container defining an interior space and an opening into the interior space, and a non-rigid sealing structure disposed and configured to seal the opening into the interior space to maintain the compressed gas under compression. The syringe includes a barrel, a syringe needle fluidly coupled to an interior of the barrel, and a plunger stopper disposed to translate within the barrel, a seal disposed to seal the barrel opposite the syringe needle. The plunger stopper is radially disposed within the barrel and separates the interior of the barrel into a drug space configured to contain the injectable drug between the plunger stopper and the syringe needle, and an actuation space between the plunger stopper and the seal. The autoinjector further includes a puncturing needle. The puncturing needle is axially aligned to selectively penetrate the non-rigid sealing structure of the compressed gas source upon relative axial movement between the puncturing needle and the compressed gas source to fluidly couple the puncturing needle with the compressed gas source. At least one of the compressed gas source and the puncturing needle is movably mounted whereby the puncturing needle selectively penetrates the non-rigid sealing structure to selectively fluidly couple the compressed gas source with the actuation space.

According to another aspect of this disclosure, there is provided a compact sealed compressed gas source. The compressed gas source includes a rigid container, a non-rigid sealing structure, a crimping sleeve, and a conically shaped rigid structure. The rigid container defines an interior space, and includes an enlarged neck portion defining an opening into the interior space. The non-rigid sealing structure is disposed and configured to seal the opening into the interior space. The non-rigid sealing structure is at least partially disposed within the opening into the opening. The crimping sleeve includes a generally cylindrical portion disposed around and crimped below the enlarged neck portion of the rigid container and a generally radially extending portion defining an aperture in alignment with the opening into the interior space. The crimping sleeve is disposed to resist outward movement of the non-rigid sealing structure from the enlarged neck. The conically shaped rigid structure is disposed to exert a sealing force against the non-rigid sealing structure. The conically shaped rigid structure may be formed by the crimping sleeve itself or by a separate structure, such as a conical washer. A compressed gas is disposed within the interior space of the rigid container.

According to further aspect of this disclosure, there is provided a method of manufacturing such a sealed compact gas source by inserting the non-rigid sealing structure into the opening into the interior space of the rigid container, disposing the crimping sleeve around the enlarged neck portion of the rigid container with the conically shaped rigid structure disposed to exert an inwardly directed sealing force on the non-rigid sealing structure, crimping the crimping sleeve around the enlarged neck portion, and charging the rigid container with a compressed gas.

According to yet another aspect of this disclosure, there is provided a method of administering an injectable drug by fluidly coupling an actuation space of a syringe with a compressed gas source to provide compressed gas to axially translate a plunger stopper within a barrel of the syringe to inject the injectable drug.

### DESCRIPTION OF THE FIGURES

FIGS. 1-1 through 1-3 are progressive side elevational schematic views of components of a partially cross-sectioned autoinjector according to teachings of this disclosure during administration of an injectable drug.

FIG. 2-1 is an exploded isometric view of the compressed gas source of FIG. 1.

FIG. 2-2 is a cross-sectional view of the assembled, compressed gas source of FIGS. 1 and 2-1.

FIG. 2-3 is an enlarged fragmentary, partially cross-sectioned view of the compressed gas source of FIGS. 1, 2-1 and 2-2.

FIG. 3 is a cross-sectional view of components of an alternative embodiment of an autoinjector according to teachings of this disclosure and an enlarged, fragmentary cross-sectional view of the autoinjector.

FIG. 4 is an exploded, isometric view of an alternative embodiment of a compressed gas source according to aspects of this disclosure, and an isometric view of the assembled compressed gas source.

FIGS. 5-1 and 5-2 are side elevational views of an autoinjector according to aspects of this disclosure, wherein the cap is in position on the housing, and the cap is removed from the housing, respectively.

FIG. 6 is an exploded isometric view of the autoinjector according to FIG. 5.

FIG. 7 illustrates side elevational views of the interiors of the front and rear housings of the autoinjector of FIGS. 5 and 6.

FIG. 8 illustrates a series of side elevational, fragmentary views of a needle safety mechanism of the autoinjector of FIGS. 5-6.

FIG. 9-1 is an isometric view of a carrier of the autoinjector of FIGS. 5-6.

FIG. 9-2 is an isometric view of the carrier of FIG. 9-1 with a compressed gas source and pinion gears of FIGS. 5-6.

FIG. 10 is an isometric view of a push rod of the autoinjector of FIGS. 5-6.

FIG. 11 illustrates the use of an injector of FIGS. 5-6, the housing being removed in FIGS. 11-2 through 11-5.

FIG. 12 is a side elevational view of an alternative embodiment of an autoinjector according to teachings of this disclosure.

FIG. 13 is an exploded isometric view of the autoinjector according to FIG. 12.

FIG. 14 illustrates a series of side elevational views of the autoinjector of FIGS. 12-13 during an injection procedure.

FIG. 15 illustrates side elevational views of the interior of the rear housing and side elevational views of the interior and exterior of the front housing of the autoinjector of FIGS. 12-14.

FIG. 16 illustrates isometric and side elevational views of the dose indicator and a fragmentary isometric view of the dose indicator disposed through a window in the front housing of the embodiment of FIGS. 12-14.

FIG. 17 is an isometric view of a relay of the embodiment of FIGS. 12-14.

FIG. 18 illustrates a side elevational view of a slider and a series of side elevational, fragmentary views of a needle safety mechanism of the autoinjector of FIGS. 12-14.

FIG. 19 illustrates a series of fragmentary view (the housing being removed) of the use of an injector of FIGS. 12-14 to deliver a dose.

FIGS. 20-1 and 20-2 illustrate fragmentary side elevational views of the autoinjector of FIGS. 12-14 prior to and at the end of injection, respectively.

FIG. 21 illustrates fragmentary side elevational views of the autoinjector of FIGS. 12-14 prior to and at the end of injection, respectively.

FIG. 22 provides a series of side elevational, schematic views of a further embodiment of an autoinjector.

FIG. 23 is a cross-sectional view of the autoinjector of FIGS. 5-6.

FIG. 24 is a cross-sectional view of the autoinjector of FIGS. 12-14.

### DETAILED DESCRIPTION OF EXEMPLARY EMBODIMENTS

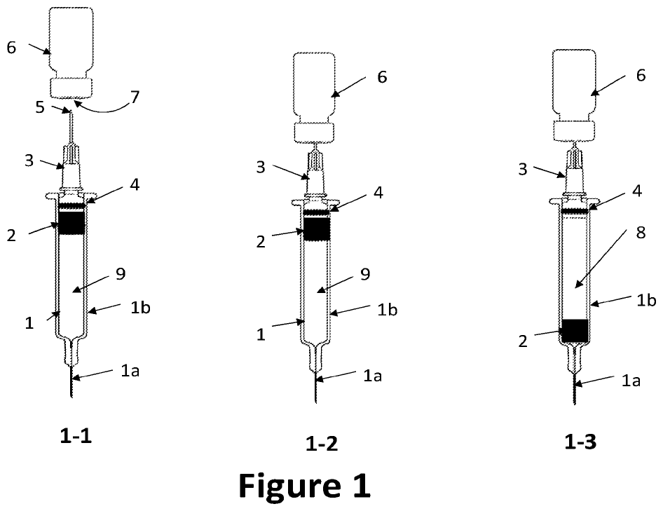

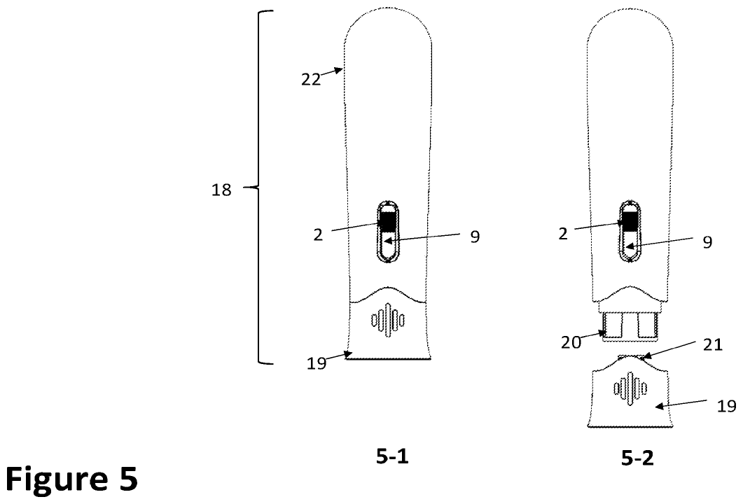

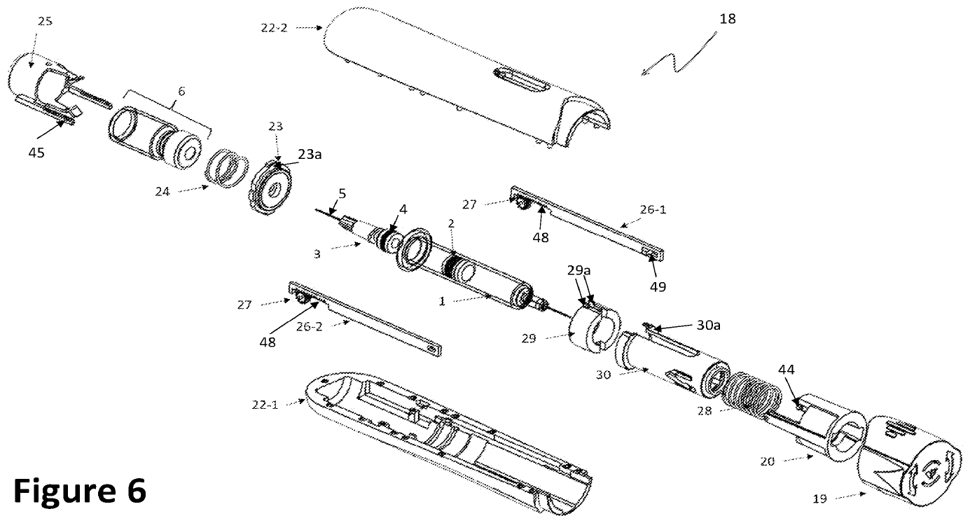

In accordance with this disclosure, there is provided an autoinjector 18 (see FIGS. 5 and 6) that includes a syringe 1 and a compressed gas source 6. FIG. 1 shows general schematic of using a compressed gas source 6 according to this disclosure to administer an injectable drug 9 contained within a syringe 1. The syringe 1 includes a needle 1a coupled to a barrel 1b through which a plunger stopper 2 is axially displaceable to administer the injectable drug 9 through the needle 1a. A needle adapter 3 is coupled to the syringe 1 opposite the needle 1a. Referring to FIG. 1-1, the plunger stopper 2 contacts the injectable drug 9 on one side and faces a needle adapter 3 on its other side. The syringe 1, plunger stopper 2 and needle adapter 3 are disposed coaxially. The needle adapter 3 is hollow, connects an axially-extending lumen of a coupled puncturing needle 5 with a space between the needle adapter 3 and plunger stopper 2. A circular seal 4 is disposed within the barrel 1b of the syringe 1, between the needle adapter 3 and the plunger stopper 2. The seal 4 isolates the lumen of the needle adapter 3 and the interior of the barrel of the syringe 1 from the external environment.

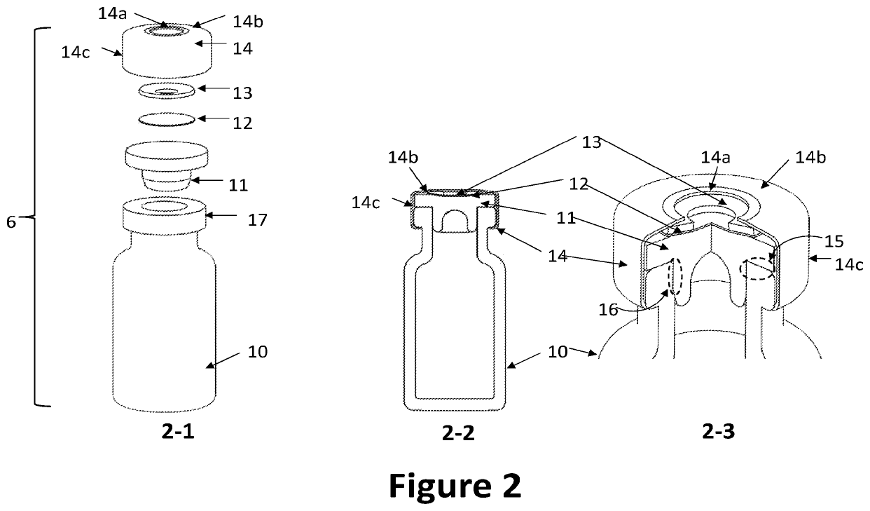

The compressed gas source 6 is in the form of a canister or vial that is generally rigid, but includes a non-rigid portion 7 that is pierceable by the puncturing needle 5. An exemplary compressed gas source 6 is explained in greater detail below with regard to FIG. 2. The compressed gas source 6 is aligned with puncturing needle 5 secured with the needle adapter 3 such that the non-rigid portion 7 of the compressed gas source 6 is directly facing a tip of the puncturing needle 5 extending from the needle adapter 3. In at least one embodiment, the size of the puncturing needle 5 would ideally be 25G or finer and have length greater than that necessary to completely pierce the non-rigid portion 7 of the compressed gas source 6. The non-rigid portion 7 consists of a non-porous (or low porosity), elastomeric (or equivalent) material. In at least one embodiment, this component may be coated or infused with an additive to further reduce its porosity.

FIGS. 1-1 through 1-3 schematically represent the coupling of a syringe 1 and needle adapter 3 with a compressed gas source 6, and the actuation of an injection. FIG. 1-1 represents the state prior to the coupling of the needle adapter 3 with the compressed gas source 6 for actuation of injection. FIG. 1-2 represents start of injection where compressed gas source container 6 axially is moved towards puncturing needle 5 such that the tip of the puncturing needle 5 penetrates past the non-rigid portion 7 of the compressed gas source 6, connecting the lumen of the puncturing needle 5 and adapter 3 with high pressure gas enclosed within the compressed gas source 6. Referring to FIG. 1-3, the compressed gas within container 6 flows through the needle adapter 3 to the space between the needle adapter 3 and plunger stopper 2. As long as the pressure of the compressed gas in the space 8 between the needle adapter 3 and plunger stopper 2 provides high enough force to overcome plunger stopper 2 gliding force and the force required to overcome resistance to fluid flow within the syringe, the plunger stopper 2 is advanced to the end of dose position as shown in FIG. 1-3.

According to an aspect of this disclosure, the rigid portion of the compressed gas source 6 is constructed of a material and have thickness capable of holding high pressure of compressed gas. The material may be stainless steel, for example, but could also be constructed from plastic as long as the rigid portion of the compressed gas source 6 is sufficiently rigid to withstand high internal pressures. Puncturing needle 5 is formed of any appropriate material, such as metal or stiff plastic. In at least one embodiment, the puncturing needle 5 may be lubricated on its outer surface.

While the disclosed compressed gas source 6 is explained and illustrated in detail with regard to the syringe 1 in the figures, those of skill in the art will appreciate that the will appreciate that the disclosed arrangement may be applied to drug delivery devices other than autoinjectors.

Those of skill in the art will further appreciate that actuation of the syringe 1 to administer an injectable drug 9 occurs when the compressed gas source 6 and the needle adapter 3 are moved relatively toward each other. That is, administration may be initiated when the compressed gas source 6 and the needle adapter 3 are physically moved toward each other, when the compressed gas source 6 is moved towards the tip of the puncturing needle 5 of a stationary needle adapter 3, or when the needle adapter 3 is moved towards a stationary compressed gas source 6.

FIG. 2 provides details of one embodiment of a structure of an exemplary compressed gas source 6. Exploded view 2-1 of the compressed gas source 6 shows rigid container 10, non-rigid part 11, pad 12, conical washer 13 and a crimping sleeve 14. The rigid container 10 is constructed to withstand pressure exerted by the compressed gas contained within it. It could be constructed using stainless steel, aluminum, or plastic, for example. The rigid container 10 includes an enlarged portion 17 defining an opening into the interior of the rigid container 10. The crimping sleeve 14 defines an aperture 14a and includes a generally cylindrical outer portion 14c that is sized to surround the enlarged portion 17. While the crimping sleeve 14 may further include a generally radially extending portion 14b that defines the aperture 14a and that extends inward from the generally cylindrical outer portion 14c, the generally radially extending portion 14b may alternatively be formed as the generally cylindrical outer portion 14c is crimped around the enlarged portion 17 of the rigid container. The non-rigid part 11 is disposed within the opening into the interior of the rigid container 10 to seal the contents. The non-rigid part 11 is soft enough to enable a needle to pass through it and compliant enough to seal against the surfaces of the rigid container 10. Pad 12 is disposed against an upper surface of the non-rigid part 11 and helps prevent bulging out of the non-rigid part when enclosing a high-pressure compressed gas. The thickness of the pad 12 is such that it is still easily pierced by a needle, but does not rip apart when opposing pressure from enclosed compressed gas. The diameter of the pad 12 is larger than both the inner diameter of conical washer 13 and the diameter of aperture of crimping sleeve 14. In at least embodiment the non-rigid part 11 is pre-disposed in within portion of the rigid container 10 having the smallest cross-sectional area.

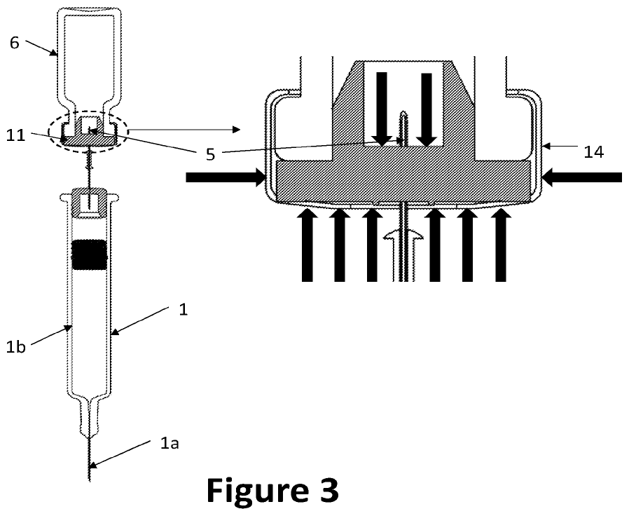

FIG. 2-2 shows a cross-sectional view of the assembled compressed gas source 6 with all the aforementioned components. Assembly is accomplished by axial compression and concurrent deformation of portion of crimping sleeve 14 in towards the axis of the rigid container 10, which secures all the components. Disposed between the crimping sleeve 14 and the non-rigid part 11 of the compressed gas source 6, it will be appreciated that the central portion of conical washer 13 is disposed to exert an axially inward force on the non-rigid portion 7 of the compressed gas source, here, the pad 12 and the non-rigid part 11. In this way, the axially inward force exerted by the conical washer 13 and the crimping sleeve 14 causes an outward force of the necked portion of the non-rigid part 11 or plug, against an interior surface of the rigid container 10, here, the interior diameter of the enlarged portion 17, at area 16. Those of skill in the art will appreciate that the crimping sleeve 14 and conical washer 13 may be formed as a unitary structure, that is, the crimping sleeve 14 could include an inner surface that oriented conically inward toward the non-rigid part 11. In an arrangement where the conical washer 13 or another conical structure of the crimping sleeve 14 exerting an inward force centrally on the non-rigid part 11 is not included and is not oriented as shown in FIG. 2-1, the non-rigid part 11 may seal against the rigid container 10 in area 15 only as shown in FIG. 2-3. This would be similar to how drug vials typically establish a seal for container closure. However, this amount of sealing may be insufficient for high pressure of compressed gas. Under compression by the crimping sleeve 14 upon assembly, the inverted conical washer 13 (or such a unitary structure) provides an angled, outward compression force to the non-rigid part 11 and enables an additional sealing in area 16. This allows retention of high pressure within the compressed gas source 6 and mitigates the risk of the gas leaking.

FIG. 3 illustrates an alternative embodiment of a needle adapter and syringe, and a compressed gas source 6. In FIG. 3, a sectioned view of embodiment is shown with partial magnified view, illustrating forces exerted on the compressed gas source 6, as well as the piercing of the non-rigid portion 7 by the puncturing needle 5 of the needle adapter 3. The arrows indicate the application of pressure on the non-rigid part 11 by both the compressed gas and the crimping sleeve 14 and washer 13. The presence of high-pressured gas and opposing pressure applied by the crimping sleeve 14 and washer 13 axially compresses the non-rigid part 11, which in turn would be squeezed radially out. However, the non-rigid part 11 is also radially constrained by crimping sleeve 14. This works to effectively reduce or eliminate porosity of the elastomeric non-rigid part 11. In addition, when a puncturing needle 5 is inserted into elastomeric non-rigid part 11, the aforementioned dynamic provides a radial compression force sealing the entry around the penetrated puncturing needle 5, and seal the compressed gas source 6 after the puncturing needle 5 is removed. In this way, the non-rigid part 11 effectively operates like a valve. Those of skill in the art will appreciate that an autoinjector embodiment may be provided incorporating a valve to control flow of compressed gas powering an injection according to the teachings of this disclosure and scope of appended claims.

In addition, as an alternative the embodiment of the compressed gas source 6 as described above, various options of modifying the design of the crimping sleeve 14 may preempt the need for washer 13 and/or the pad 10.

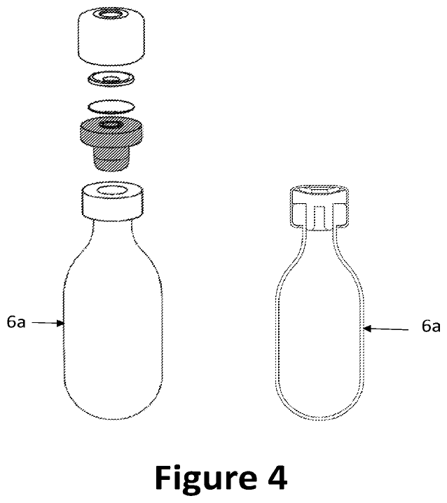

In at least one embodiment, the sealing surfaces are highly polished to ensure a good seal. The form factor of the compressed gas source 6 can be modified to accommodate higher pressures—for e.g., the rigid container 6a may be provided with a hemispherical bottom (see FIG. 4), etc. Those of skill in the art will appreciate that modifications may likewise be provided to facilitate filling, including incorporation of a valve. In at least one embodiment, the compressed gas source 6 may be filled with pressurized gas using a needle, which is angled into the non-rigid part 11 for filling and removed by retracing its entry path. However, other methods to fill compressed gas or liquid phase gas without use of a needle piercing the non-rigid part 11 are likewise envisioned by this disclosure.

FIG. 5 shows one embodiment of an autoinjector 18 incorporating aforementioned compressed gas source 6. FIG. 5-1 illustrates the autoinjector embodiment 18 having housing 22 and cap 19. The housing 22 may include a window such the injectable drug 9 and syringe plunger stopper 2 are visible through the window in the housing 22. Removal of cap 19, as illustrated in FIG. 5-2, exposes a needle safety shield 20, which obscures view of the needle contained within the autoinjector 18. Removal of the cap 19 also removes the needle cover 21.

According to a feature of at least one embodiment, actuation of injection is accomplished by full retraction of needle safety shield 20. Once the injectable drug 9 is completely delivered and the user removes the autoinjector 18 away from the injection site, the safety shield 20 locks out preventing accidental needle-stick injury from a biohazardous injection needle.

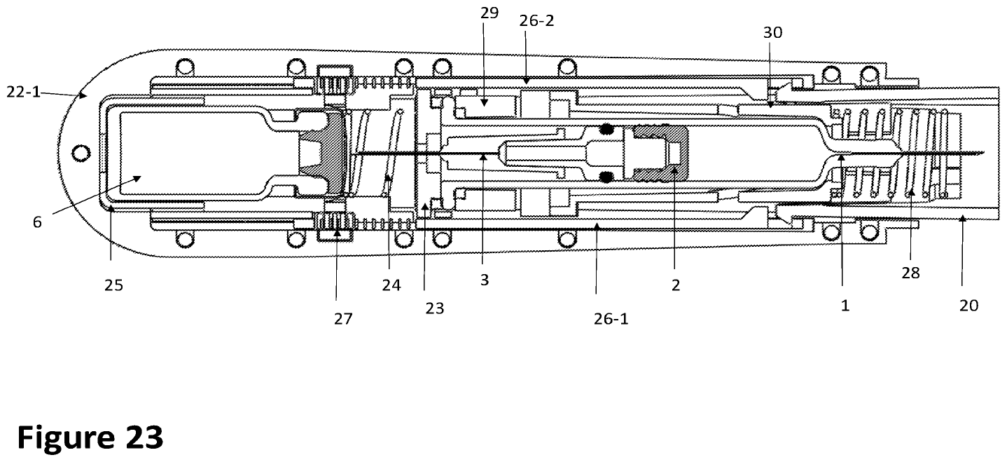

FIG. 6 is an exploded view of the autoinjector 18 showing various components contained within the autoinjector 18, while a cross-sectional view of the assembled autoinjector is provided in FIG. 23. Longitudinally split housings 22-1 and 22-2 enclose constituent components of autoinjector 18. While illustrated embodiment shows housing longitudinally split, it is possible to design the housings to be split transverse to the axis of the autoinjector 18 for manufacturability purposes. The compressed gas source 6 is contained in carrier 25 and is biased away from needle adapter 3 by a biasing element, here, spring 24. The spring 24 is axially supported by disc 23, which also acts as an axial stop for syringe 1. The disc 23 includes a centrally disposed lumen to allow for passage of the needle adapter 3.

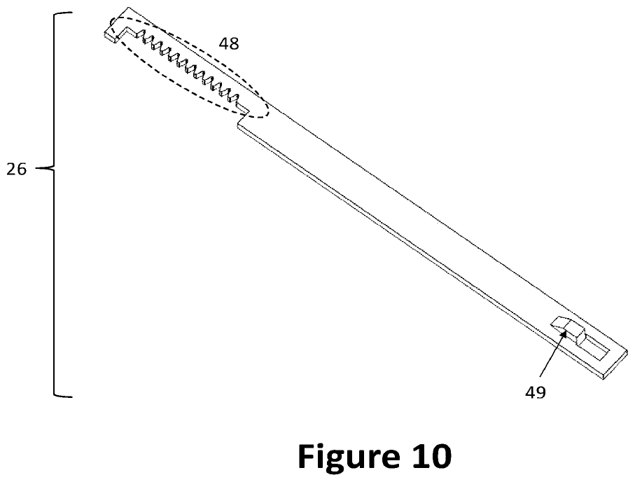

The syringe 1 is coupled to the housing 22 using clip 29, which has tabs threading into features 32 on housings 22-1 and 22-1 once closed. Slider 30 encloses outer surface of the syringe 1 and is configured to rotate around the axis of syringe 1. Slider 30 includes features on that help retain needle safety shield 20, which is biased away from the slider 30 by safety spring 28. As will be explained in greater detail with regard to FIG. 10, push rods 26-1 and 26-2 transfer motion from the safety shield 20 to carrier 25 via racks 48 engaging with pinion gears 27. Cap 19 helps close the autoinjector 18, and also contains a feature such that removal of the cap also removes needle shield of the syringe 1. Hence, once the cap 19 is removed, the autoinjector 18 is ready to inject.

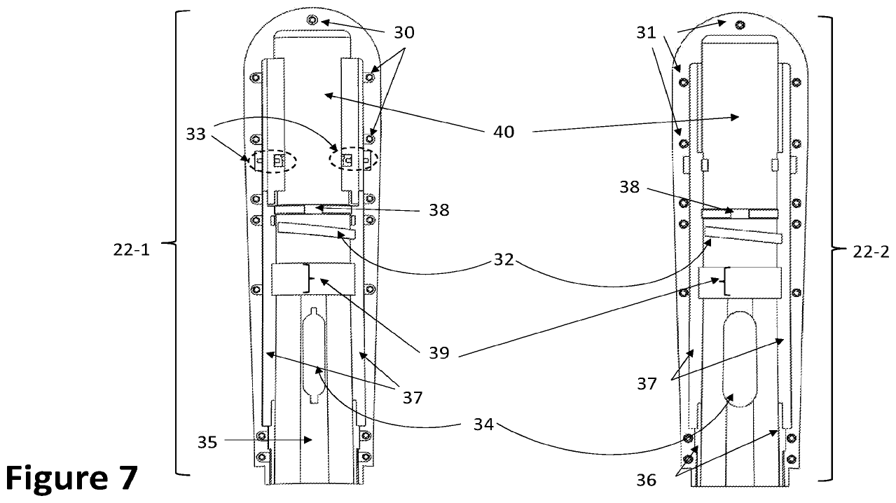

FIG. 7 shows inside features of back housing 22-1 and front housing 22-2. The two housings can be attached to close the autoinjector device by press fitting cylindrical posts 31 into slightly undersized hexagonal holes 30. Track 32 provides thread for tabs 29a on clip 29 to secure syringe 1. As tabs 29a ride in track 32, the clip 29 rotates relative to the housing 22. Features 33 facilitate placement of pinion gears 27 of push rods 26 within the housing, and relative to the engaging components. Windows 34 facilitate inspection of the injectable drug 9 contained in syringe 1 mounted within the back and front housings 22-1, 22-2. Tracks 35 and 36 are configured as axial keying features for elements of the needle safety shield 20. Slots 37 are configured to receive push rods 26-1 and 26-2, allowing for axial movement of the push rods 26-1, 26-2 as a result of rotation of the pinion gears 27 mounted at 33 and their engagement with racks 48. Slot 38 provides axial and rotational retention of disc 23, protrusions 23a of the disc 23 being received within the slots 38. Cylindrical slots 39 on both housings provides axial retention of slider 30, the cylindrical slots 39 being configured to receive flange 30a of the slider 30. Cavity 40 is configured to receive carrier 25.

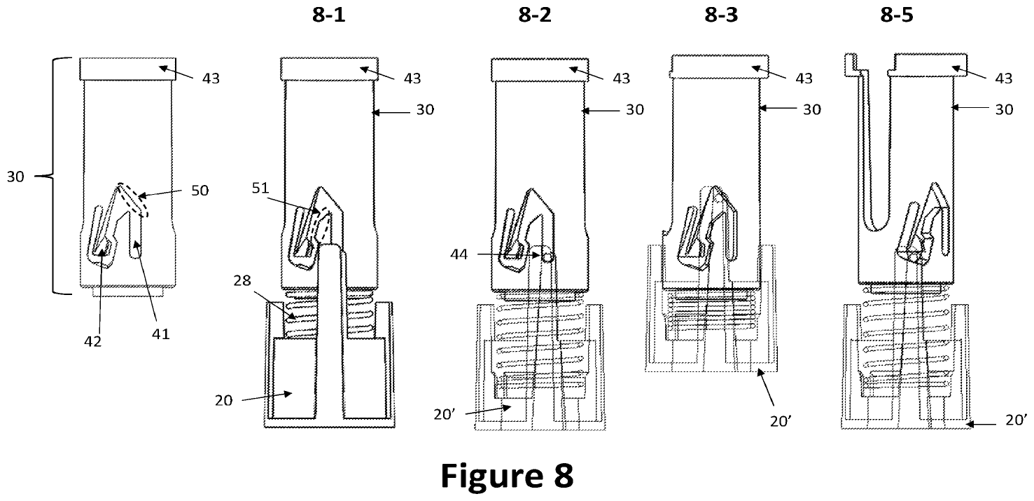

Turning now to FIG. 8, there is illustrated a needle safety mechanism, which in this embodiment operates independently of the drug dosing mechanism. Slider 30 is a hollow cylindrical structure which includes a protruding cylindrical flange portion 43; in assembly, the flange portion 43 is received within slot 39 of the housings 22-1, 22-2 to axially constrain the slider 30, yet permit rotation of the slider 30 relative to the housing 22. The slider 30 further includes at least one track 41 within an exterior wall of the slider 30. The needle safety shield 20 includes at least one pin 44 extending radially inward. The track 41 is configured to allow the at least one pin 44 of the needle safety shield 20 to translate along a predetermined path to control the position of the safety shield relative to the slider 30.

Locking beam 42 ensures at the needle safety shield 20 cannot retract after the injection procedure is complete-hence, preventing accidental needle stick injury. FIG. 8-1 shows a safety spring 28 between slider 30 and needle safety shield 20. In at least one embodiment, at least a portion of the needle safety shield 20′ is transparent in order to allow and operator to visualize operation of needle safety module (see FIG. 8-2).

The radially inward facing pin 44 of the needle safety shield 20 is disposed within, and is configured to ride in the track 41 in order to retain the needle safety shield 20′ despite compressed safety spring 28, and to control the positions of the needle safety shield 20 and the slider 30 relative to one another. Upon initial removal of the cap 19 from the autoinjector 18, the needle safety shield 20 and the slider 30 are in the position illustrated in FIGS. 8-1 and 8-2. As the autoinjector 18 is pressed axially against a surface to allow the safety shield 20 to retract, the pin 44 traverses vertically along track 41. As the pin 44 confronts surface 50, surface 50 guides the pin 44 the position illustrated in FIG. 8-3 causing a rotation of the slider 30 relative to the needle safety shield 20. This would be similar to a situation where needle is inserted into injection site. Since the needle safety shield 20′ is axially keyed to and rotationally constrained by tracks 35 and 36, the slider 30 turns slightly with pin 44 guided by surface 50. Once the injection is complete and needle is removed from the injection site, the pin 44 takes different path guided by surface 51 to a position below locking beam 42. Again, since the needle safety shield 20′ is axially keyed to and rotationally constrained by tracks 35 and 36, the slider 30 turns further. That is, an axially exerted separating force exerted by the spring 28 may exert a separating force between the needle safety shield 20 and the slider 30. As the pin 44 rides downward in the track 41, however, the pin engages and rides along surface 51 of the track 41, further rotating the slider 30 relative to the needle safety shield 20 such that the pin cannot return to its position in FIG. 8-2. Rather, the slider 30 and the needle safety shield 20 move the relative positions illustrated in FIG. 8-5 as the spring 28 continues to exert a separating force. A portion of the slider 30 may be visible or completely obscure the window 34 at this point. This provides visual confirmation that the device has been used. The height of the pin 44 is ideally equal to the thickness of walls of slider 30.

While the sliding and locking arrangement has been described with regard to a pin 44 disposed to move within a track 41 in slider 30, it will be appreciated by those of skill in the art that alternate arrangements may be provided for controlling axial movement of the needle safety shield 20 between a shielded or safety position wherein the syringe needle 1a is not axially exposed to an injection position wherein the syringe needle 1a is axially exposed for injection. By way of example only, a track may be provided along an inner surface of the housing 22, with the pin extending radially outward from the needle safety shield 20.

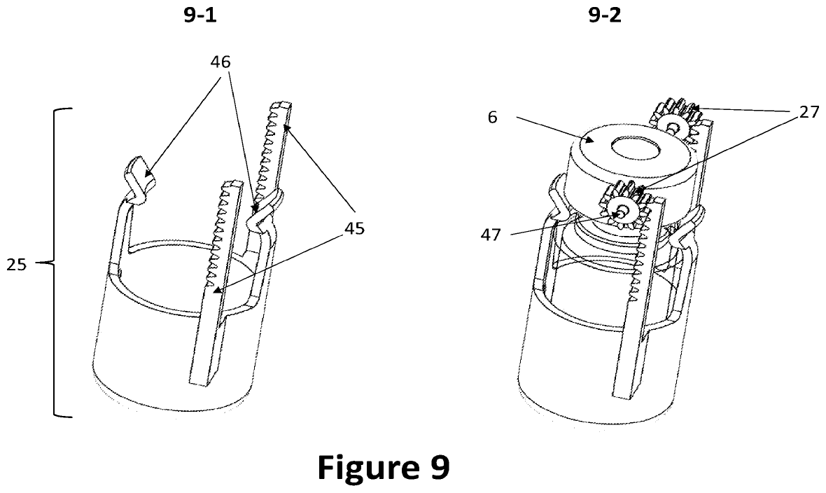

Turning now to the retention of the compressed gas source 6 within the housing 22, carrier 25 is shown in FIGS. 9-1 and 9-2 includes retaining features 46 that are disposed and configured to retain the compressed gas source 6. In order to facilitate axial translation of the compressed gas source, the carrier 25 further includes at least two linear racks 45 that have teeth meshing with the pinion gears 27. The pinion gears 27 rotate about pins 47, which are placed in features 33 of back housing 22-1.

One of the axial keying features of needle safety shield 20 is axially aligned with protrusion 49 of push rod 26 (shown in FIG. 10). Racks 48 on the push rods 26 mesh with teeth of the pinion gears 27 diametrically across from racks 45. This arrangement ensures that the push rod 26 and carrier 25 always axially translate in opposite directions from each other. Hence, retraction of needle safety shield 20 retracts the push rod 26, which in turn causes the carrier 25 to advance the compressed gas source 6 towards the needle adapter 3, which in turn drives administration of the enclosed drug 9.

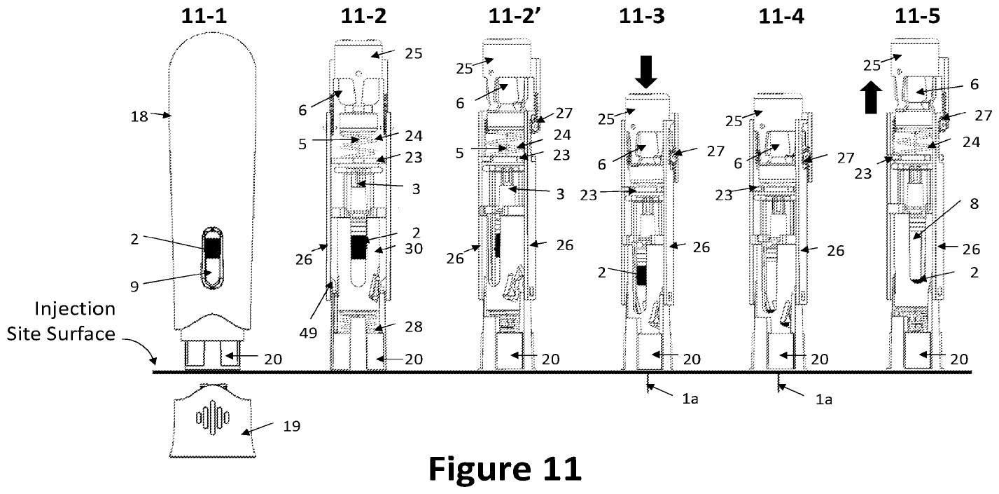

The sequence of steps from start to end are illustrated in FIG. 11. FIG. 11-1 shows autoinjector 18 with drug 9 and plunger stopper 2 visible, and its cap 19 removed, the needle safety shield 20 contacting the surface of the injection site. FIG. 11-2 is the same orientation of autoinjector 18 without housings 22-1 and 22-2. Here shown is how the axial keying feature of needle safety shield 20 is axially aligned with protrusion 49 of push rod 26. The safety spring 28 is placed in slight compressed state between needle safety shield 20 and slider 30. This is the “ready to inject” position. FIG. 11-2′ is the same “ready to inject” position, but the autoinjector is rotated about its axis slightly for better visibility of the constituent components when explaining the operation of the device. Spring 24 in this state biases the compressed gas source 6, which is disposed in carrier 25 away from the tip of the puncturing needle 5 in the needle adapter 3. When an axial force shown by the arrow in FIG. 11-3 is applied by the user, the needle safety shield 20 pushes push rod 26 in a direction opposite to the illustrated arrow, which rotates pinion gear 27 in a clockwise direction as shown. This in turn causes the carrier 25 and hence the compressed gas source 6 to be advanced to the tip of the puncturing needle 5 of needle adapter 3 and eventually piercing the non-rigid part 11 of the compressed gas source 6. The spring 24 disposed between the compressed gas source 6 and the disc 23 is now compressed. Similarly, the spring 28 disposed between the needle safety shield 20 and the slider 30 is compressed as the needle 1a of the syringe 1 enters the injection site, as illustrated in FIG. 11-3. The plunger stopper 2 now moves from start of dose position in FIG. 11-3 to the end of dose position in FIG. 11-4, with the needle 1a of the syringe 1 is at the injection site under the surface of the skin.

After drug 9 is completely delivered, the autoinjector 18 is removed from the injection site in direction of the arrow in FIG. 11-5. Spring 24 and safety spring 28 act in unison to passively lock the needle safety shield 20 (described previously and illustrated in FIG. 8). In addition, the compressed gas source 6 is separated from the tip of the puncturing needle 5 of the needle adapter 3. The high pressure in the chamber 8 behind the plunger stopper 2 is now released through the tip of the puncturing needle 5 of the needle adapter 3.

Because the unique self-sealing property of the non-rigid part 11 of the compressed gas source 6, this embodiment enables relief of high pressure from the syringe 1 chamber after drug delivery is complete. If necessary, depressurizing the compressed gas source 6 after drug delivery may also be implemented.

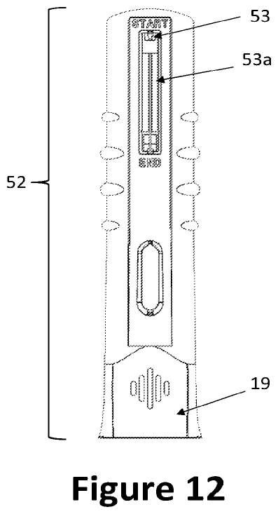

Shown in FIG. 12 is second embodiment of an autoinjector device 52 incorporating the compressed gas source 6. This embodiment of the autoinjector 52 differs from previous embodiment of the autoinjector 18 in that the compressed gas source 6 is stationary. Also, in embodiment of the autoinjector 52, a dose indicator 53 is implemented by tethering it to plunger stopper 2. Also, unique to embodiment 52 is how the tethered indicator 53 actuates needle safety only close to end of dose delivery by slightly modifying slider 30 from the embodiment of autoinjector 18.

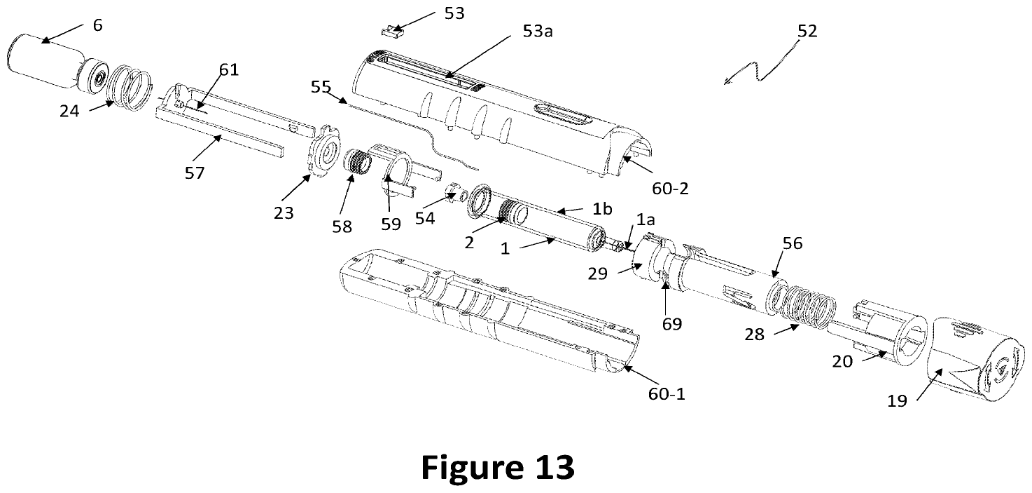

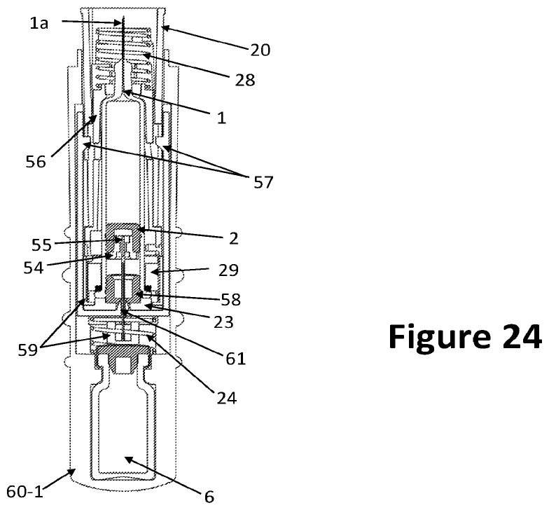

FIG. 13 is an exploded view of constituent components of embodiment 52, while a cross-sectional view is provided in FIG. 24. Several components of embodiment 52 are common to embodiment 18. All the components are enclosed within back housing 60-1 and front housing 60-2. Front housing 60-2 also has a window 53a through which dose indicator 53 is visible. A clear cover (not shown) for window 53a may optionally be included. Front housing 60-2 can have various visual cues inscribed (or printed) on it indicating status of dose delivery vis axial position of dose indicator 53. This dose indicator 53 is connected via a tether 55 to adapter 54, which in turn is secured to plunger stopper 2. The adapter 54 may be coupled to the plunger stopper 2 by any appropriate arrangement. For example, the adapter 54 may be threaded into plunger stopper 2 or have barbs for secure attachment to plunger stopper 2. The adapter 54 may also consist of an O-ring to seal against the inside surface for the syringe 2; in at least some embodiments, this would preempt the need for physical attachment to the plunger stopper 2. The connection of tether 55 to the adapter 54 and/or the dose indicator 53 may be by any appropriate arrangement. For example, such coupling may be provided by crimping or welding or insert molding to ensure secure attachment. Once assembled as a taut tether 55, motion of dose indicator 53 is synchronous with plunger stopper 2.

Syringe 1 has a staked needle 1a and contains an injectable drug 9. A slider 56 is concentric with syringe 1 and can rotate around the syringe 1 axis. The slider 56 has features that engage a coaxially placed needle safety shield 28, spring 28 being disposed between them. The slider 56 is axially constrained by features of housings 60-1 and 60-2, and also by the bottom shoulder of the syringe 1. As with the first embodiment, however, the slider 56 is rotatable relative to the axis of the syringe 1. Slider 56 also has features to engage a relay 59, which transmits linear travel of the indicator 53 to facilitate rotation of slider 56. A push rod 57 is axially keyed to the needle safety shield 20. The push rod 57 transmits linear motion to actuate dose delivery from retraction of needle safety shield 20 to puncturing needle 61. Prior to injection (or in the state as received by the user), one tip of the puncturing needle 61 is pointing at, but external to the compressed gas container. The other tip of puncturing needle 61 is embedded just past an adapter 58 inside of syringe 1 on the non-drug contacting side of plunger stopper 2. The adapter 58 can be one elastomeric component or multiple components containing an elastomer creating a seal between the tip of puncturing needle 61 and the inner surface of the syringe 1. The adapter 58 is axially fixed by disc 23. The puncturing needle 61 and the push rod 57 may be either axially keyed with each other or made as one component by insert molding puncturing needle 61 into the push rod 57. A biasing element such as spring 24 biases the push rod 57 (and hence also puncturing needle 61) away from the compressed gas source 6.

Cap 19 may be designed to be flush with housings 60-1 and 60-2. It also has engaged with needle cap of syringe 1 such that the needle is exposed when the cap 19 is removed.

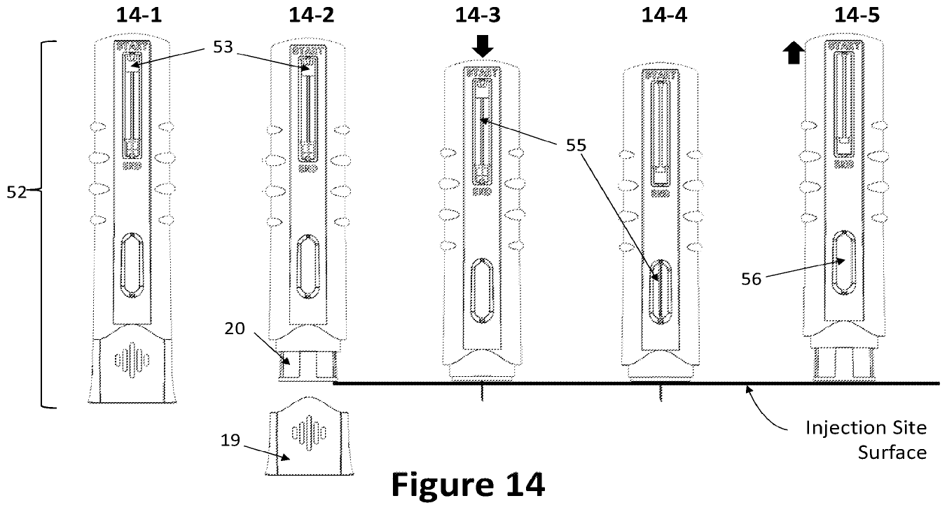

FIG. 14 shows various steps of operation of autoinjector 52. Removing cap 19 exposes needle safety shield 20. In at least one embodiment, the dose indicator 53 is disposed at a “START” sign inscribed on front housing 60-2. When the autoinjector 52 is pushed in the direction of the arrow towards the injection site surface, the dose indicator 53 travels from the “START” position in 14-3 to the “END” position in 14-4; this happens synchronously with movement of the plunger stopper 2 (not visible), which is connected to dose indicator 53 with tether 55. When the device embodiment 53 is pulled away from the injection site surface as shown in 14-5, portion of the slider 56 is visible obscuring view of the syringe 1 and its contents. Those of skill in the art will appreciate that the surface design of slider 56 may be modified to enable viewing of syringe 1 contents corresponding to 14-5.

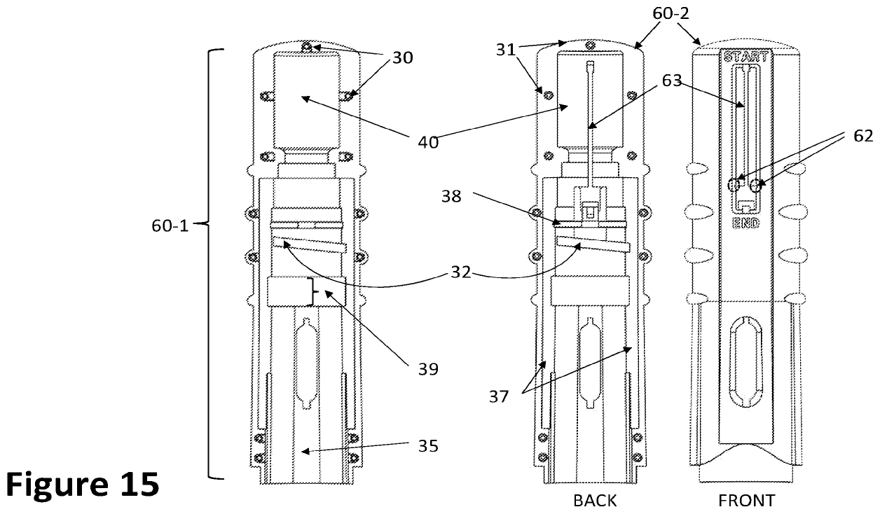

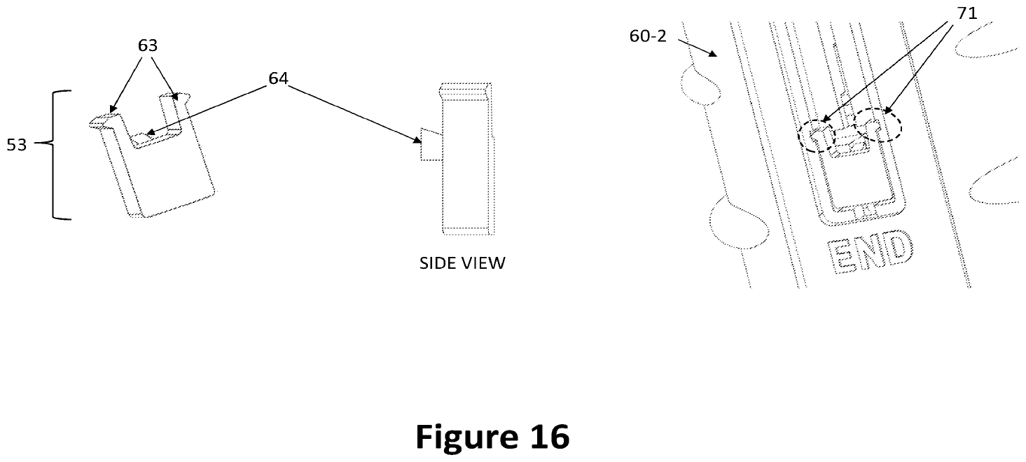

FIG. 15 shows front housing 60-2 and back housing 60-1 of the autoinjector 52. Several features of the inside of both housings 60-2 and 60-1 are identical in function to those of front housing 22-2 and back housing 22-1, and hence labelled as such. Longitudinal slit 63 provides a track for protrusion 64 (see FIG. 16) of dose indicator 53 to traverse from start to end of dose. Features 62 enable beams 63 (see FIG. 16) of the dose indicator 53 to click into indents 71 (see FIG. 16) on the front housing 60-2. The number and pattern (spacing) of the indents may be altered to create pattern of click(s) for more discriminating audible indicator(s).

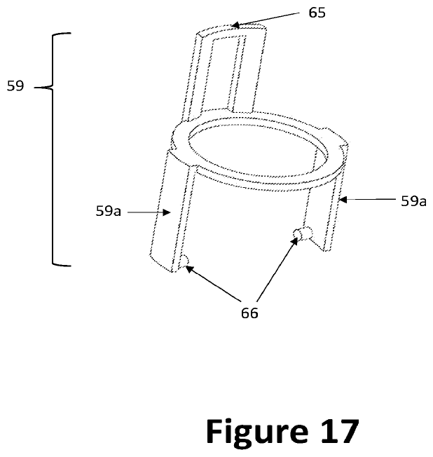

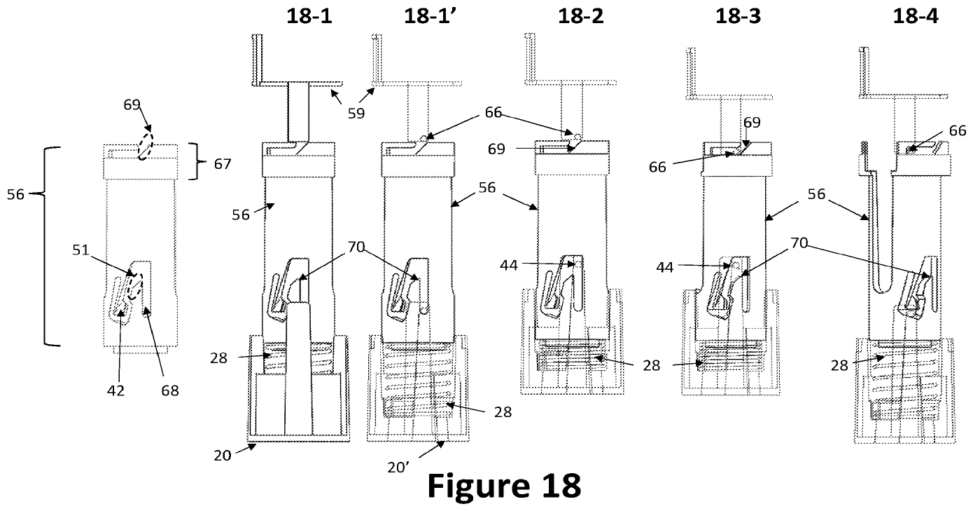

Relay 59 is shown in FIG. 17. The flat portion of protrusion 64 of the dose indicator 53 impacts surface 65 of the relay 59 close to end of dose. Pins 66 extending axially inwardly from the longitudinal members 59a of the relay 59 articulate along ramp 69 of slider 56 (shown in FIG. 18). Slider 56 differs somewhat from slider 30 of the autoinjector 18 of the first embodiment. More specifically, the ramp 41 on slider 30 of the autoinjector 18 guides pin 44 past point of no-return 70, whereas track 68 of the slider 56 of the autoinjector 52 by itself does not guide pin 44 past point of no return 70. The effect of this is that despite multiple retractions of the safety needle shield 20, the needle safety shield 20 locking mechanism would not be actuated in embodiment 52 unless pin 44 is guided past the point of no return 70. This requires the slider 56 to be rotated, which may be achieved by ramp 69 and pin 66 of the relay 59.

Turning now to FIG. 18, there is illustrated a series of side elevational, fragmentary views of a needle safety mechanism of the autoinjector 52. FIGS. 18-1 and 18-1′ show positions of various components illustrated as received by the user. As the autoinjector 52 is depressed against a target surface, the user retracts the needle safety shield 20 as shown in 18-2. When end of dose delivery occurs, the pin 66, which is axially keyed, rides on ramp 69 of slider 56. Because the position of the slider 56 is axially fixed by feature 67 within slot 39 of the housings 60-1 and 60-2, it is forced to rotate as pin 66 is driven axially by the dose indicator 53 (not shown). This rotation takes pin 44 past the point of no return as shown in FIG. 18-3. In 18-4, as spring 28 biases the needle safety shield 20 away from the slider 56, the pin 44 of the needle safety shield 20 is guided down ramp 51 passively (without user effort) by the force provided by spring 28 to eventually be placed below locking beam 42.

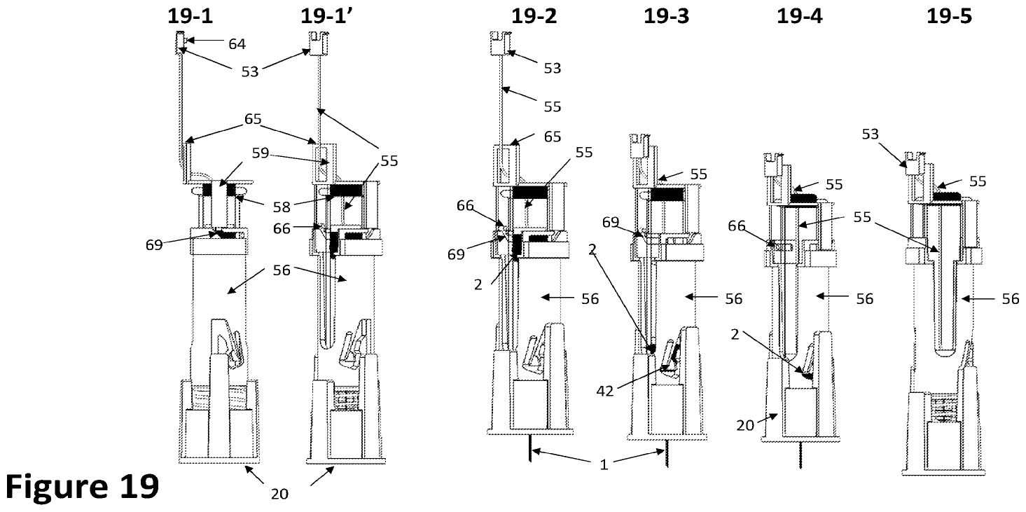

FIG. 19 shows various stages of interplay between the dosing mechanism and the needle safety mechanism. FIGS. 19-1 and 19-1′ are different angular views of the components prior to start of dose delivery. FIG. 19-2 shows the needle safety shield 20 retracted as a result of an axial force applied to move the autoinjector 52 toward an injection site, triggering the start of injection moving plunger stopper 2 from start of dose position in FIG. 19-2 to close to end of dose position in FIG. 19-3. At this time, the protrusion 64 of dose indicator 53 impacts surface 65 of relay 59. The plunger stopper 2, which is driving motion of dose indicator 53 via the tether 55, reaches end of dose position illustrated in FIG. 19-4. Pin 66 of the relay 59 is now at the bottom of ramp 69 of the slider 56. In FIG. 19-5, translation of pin 44 down the ramp 51 of slider 56 as the needle safety shield is locked out once total allowed spring 28 expansion is realized.

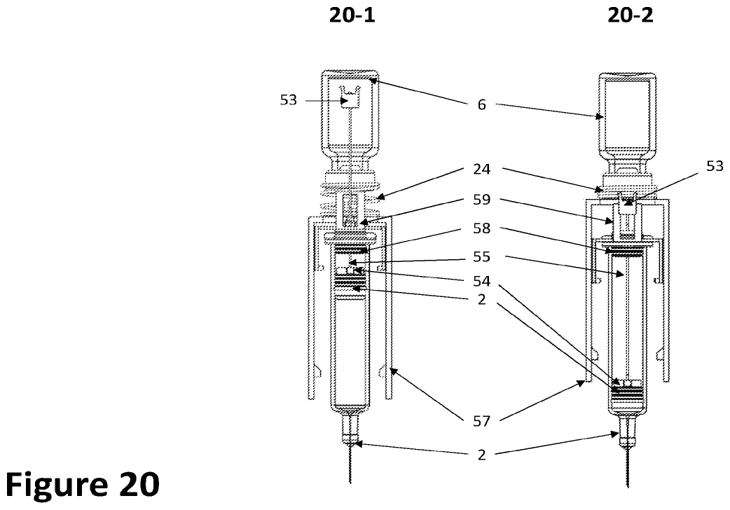

FIG. 20 shows select components in prior to the start of injection (FIG. 20-1) and end of dose delivery (FIG. 20-2). In order to actuate the autoinjector 52 to start an injection, the push rod 57 is pressed vertically against spring 24 by retracting needle safety shield 20 (not shown). This causes puncturing needle 61 (not visible in FIGS. 20-2 and 20-2), which is axially keyed to the push rod 57, to insert its tip into the non-rigid part 11 of the compressed gas container 6. This provides a path for the pressurized gas to travel through puncturing needle 61 directly into the syringe 1. Axially stationary seal 58 ensures that this compressed gas is directed solely to advance plunger stopper 2 towards the end of dose position. Since plunger stopper 2 is tethered to the dose indicator 53, the dose indicator synchronously moves with the plunger stopper 2 towards the end of dose position. The entry (pierced or circumferential seal) of both the tether 55 and puncturing needle 61 are sealed by seal 58.

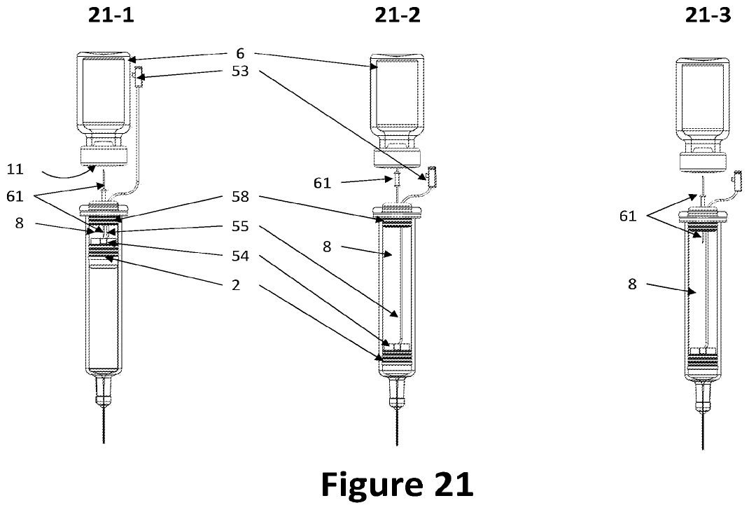

FIG. 21 further illustrates transfer of compressed gas for drug injection. FIG. 21-1 represents stage prior to start of injection. One of the puncturing needle 61 tips is placed between seal 58 and plunger stopper 2 in space 8. In FIG. 21-1, space 8 is at atmospheric pressure. The other puncturing needle 61 tip is pointing to the compressed gas source spaced away from non-rigid part 11 of the compressed gas container 6. When the puncturing needle 61 tip is advanced to go past the non-rigid part 11 of the compressed gas container 6 by retraction of the needle safety shield 20 (not shown), the compressed gas is provided a path to space 8, thereby advancing plunger stopper 2 to complete the injection and also as shown in FIG. 21-2, the dose indicator 53 is fully advanced at end of dose. When the needle safety shield 20 is separated from the injection site, the push rod 57 (not illustrated in FIG. 21-3) moves puncturing needle 61 away from the non-rigid part 11 of the compressed gas container 6. This causes pressurized gas to be released from chamber 8 through the now unsealed puncturing needle 61 tip on side of the compressed gas container 6.

As outlined previously, the needle safety shield 20 does not lock out until end of dose is reached in autoinjector 52. This means that if the user removed embodiment 52 away from the injection site prior to completion of the injection, the safety shield 20 would not be locked, but the compressed gas in chamber 8 would be released stopping (interrupting) the injection. When the user re-inserts the injection needle retracting the needle shield 20, the injection is resumed by reintroduction of pressurized gas flow by reestablishing connection between the compressed gas container 6 and space 8. Since some of the pressure was released when the injection was interrupted, it is envisioned that remainder of injection will occur at lower flow rate. While multiple interruptions of injection are possible, pausing injection may be only a last recourse. The unique ability to pause injection as outlined here may be beneficial for new patients unfamiliar with use of an autoinjector. This unique feature to pause and resume injection using an autoinjector ensures that a mistake may not result in waste of the drug. There may be multiple other benefits also. The ability to pause injections is not known to be implemented in a non-electronically powered autoinjector. To the best of our understanding in the state of art for non-electronically powered autoinjectors, should the user remove the autoinjector (accidentally or otherwise) from the injection site, the device continues to expel the drug into the environment and hence waste the drug. A wasted dose is a missed dose. Even with this feature to pause the injection, however, some minimal loss of drug may occur due to inertia.

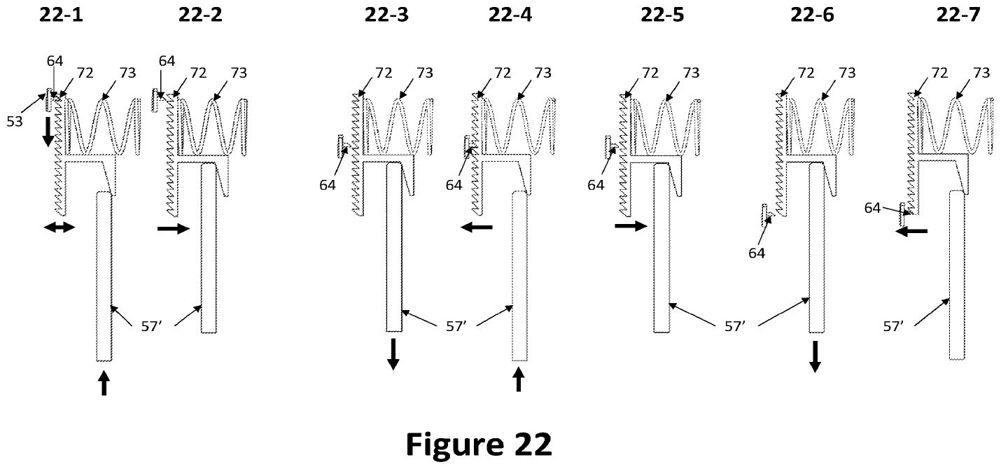

In FIG. 22, another embodiment to enable pausing of the injection in an autoinjector is illustrated. In case of a gas-powered autoinjector, this embodiment enables pausing of the injection without loss of pressure. The schematic shows dose indicator 53, which may be, tethered to plunger stopper 2 or a component abutting it (only dose indicator 53 is illustrated). A ratchet stop 72 has sawtooth pattern and is biased by a spring 73. The flats of the sawtooth pattern face the flat side of protrusion 64 of the dose indicator 53. In this embodiment, a power source would drive the dose indicator 53 via its tethering to plunger stopper 2 or component abutting the plunger stopper 2, but the dose indicator 53 advancement is prevented by ratchet stop 53 as illustrated in 22-1. When the push rod 57′ is fully retracted by the needle safety shield 20 (not shown), the ratchet stop 72 is pushed away orthogonally to the direction of motion of dose indicator 53. The dose indicator 53 is thereby released, allowing the injection to proceed as shown in 22-2. However, when the injection is interrupted by removing the autoinjector away from the injection site, this causes the axially keyed push rod 57′ to move in the direction as shown in 22-3. As a result, the saw tooth ratchet stop 72 is re-engaged with the dose indicator 53 as shown in 22-4. One more pause of injection through end of injection is illustrated in 22-5 through 22-7.

It is envisioned that this aforementioned scheme could be adapted in a spring-powered autoinjector.

Those of skill in the art will appreciate that other arrangements for locking out needle safety shield at the end of injection may be accomplished based upon the teachings of this disclosure. For example, a cam-based mechanism can be implemented incorporating concepts disclosed herein.

The autoinjector housings can be re-configured to split transversely to the axis of the device instead of the longitudinal split housing design to enable better manufacturability and assembly with a prefilled syringe.

It is envisioned that the slider in either embodiment could be placed coaxially with the syringe without an axial overlap, and yet result in the same outcome as articulated in the foregoing.

Those of skill in the art will appreciate that the length of tether 55 may be controlled in order to apply teachings of this disclosure to a variable dosing autoinjector embodiment in order to enable the user to set a dose prior to the injection.

Incorporation of electronic communication components and use of the disclosed invention with electronic methods of data capture, management and transmission are envisioned as part of this disclosure.

It will be appreciated that the foregoing description provides examples of the disclosed autoinjectors and techniques. However, it is contemplated that other implementations of the disclosure may differ in detail from the foregoing examples. All references to the disclosure or examples thereof are intended to reference the particular example being discussed at that point and are not intended to imply any limitation as to the scope of the disclosure more generally. All language of distinction and disparagement with respect to certain features is intended to indicate a lack of preference for those features, but not to exclude such from the scope of the disclosure entirely unless otherwise indicated.

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context.

Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

[We/I] claim:

1. An autoinjector for use in the injection of an injectable drug with assistance of a compressed gas, the autoinjector comprising:
a compressed gas source including a rigid container defining an interior space and an opening into the interior space, and a non-rigid sealing structure disposed and configured to seal the opening into the interior space to maintain the compressed gas under compression;
a syringe including a barrel, a syringe needle fluidly coupled to an interior of the barrel, and a plunger stopper disposed to translate within the barrel, the plunger stopper radially disposed within the barrel, the plunger stopper separating the interior of the barrel into a drug space configured to contain the injectable drug between the plunger stopper and the syringe needle, the syringe further including a seal disposed in contact with the barrel to form an actuation space within the barrel between the plunger stopper and the seal, the actuation space being in fluid communication externally to the syringe through the seal;
a housing mounting the compressed gas source and the syringe relative to one another; and
a hollow puncturing needle axially aligned to selectively penetrate the non-rigid sealing structure of the compressed gas source upon relative axial movement between the puncturing needle and the compressed gas source to fluidly couple the puncturing needle with the compressed gas source, at least one of the compressed gas source and the puncturing needle being movably mounted whereby the puncturing needle selectively penetrates the non-rigid sealing structure to selectively fluidly couple the compressed gas source with the actuation space through the puncturing needle.

2. The autoinjector of claim 1 further including a needle safety shield, the needle safety shield being slidably disposed relative to the syringe whereby an axial force exerted on the needle safety shield slides the needle safety shield relative to the syringe from a shielded position wherein the syringe needle is not axially exposed to an injection position wherein the syringe needle is axially exposed for injection.

3. The autoinjector of claim 2 wherein the needle safety shield is configured to slidably return to the shielded position when the axial force is removed.

4. The autoinjector of any of claims 2-3 wherein movement of the needle safety shield relative to the syringe initiates relative movement between the puncturing needle and the compressed gas source.

5. The autoinjector of claim 4 wherein the needle safety shield is linked to the compressed gas source such that axial movement of the needle safety shield results in a corresponding axial movement of the compressed gas source in a direction axial opposite to the direction of axial movement of the needle safety shield whereby the compressed gas source moves toward the puncturing needle.

6. The autoinjector of claim 4 wherein the needle safety shield is linked to the puncturing needle such that axial movement of the needle safety shield results in a corresponding axial movement of the puncturing needle toward the compressed gas source whereby the compressed gas source moves toward the puncturing needle.

7. The autoinjector of claim 3 including a biasing element disposed to slidably return to the shielded position when the axial force is removed.

8. The autoinjector of claim 3 wherein the needle safety shield becomes axially locked in the shielded position when the axial force is removed.

9. The autoinjector of any of claims 2-8 further including a slider, the slider being rotatably disposed relative to at least the syringe, the movement of the needle safety shield is at least partially controlled by a pin and track arrangement, one of the pin and the track being formed with the needle safety shield and the other of the pin and the track being formed with the slider whereby movement of the pin within the track controls rotation of the slider.

10. The autoinjector of claim 9 including at least one biasing element, and wherein the track includes a point-of-no-return, the pin returning the needle safety shield to the shielded position when the axial force is removed when the pin is position in the track proximal to the point-of-no-return, and the pin moving to and locking the needle safety shield in the shielded position when the axial force is removed when the pin is position in the track distal to the point-of-no-return.

11. The autoinjector of claim 10 wherein the puncturing needle is removed from the compressed gas source when the pin is position in the track proximal to the point-of-no-return and the axial force is removed from the autoinjector, such that a second axial force may be applied to the autoinjector to provide a subsequent injection from the syringe.

12. The autoinjector of any of claims 9-11 further including a member coupled to the plunger stopper, the position of the member being indicative of the position of the plunger stopper within the barrel, rotation of the slider being actuated by movement of the member coupled to the plunger stopper.

13. The autoinjector of claim 12 wherein the member coupled to the plunger stopper is a dose indicator.

14. The autoinjector of any of claims 1-13 wherein the puncturing needle is fluidly disengaged from the compressed gas source upon conclusion of an injection while remaining fluidly coupled to the actuation space whereby remaining compressed gas within the actuation space is released to atmosphere.

15. The autoinjector of any of claims 1-14 further including a biasing element disposed between the puncturing needle and the compressed gas source whereby the puncturing needle is biased away from the compressed gas source, the biasing element moving the puncturing needle out of engagement with the compressed gas source when an axial force is discontinued upon conclusion of an injection.

16. The autoinjector of any of claims 1-15 further including a hollow adapter, the adapter being fluidly coupled with the puncturing needle and the actuation space through an opening in the seal, whereby the selectively fluidly coupling the puncturing needle with the compressed gas source fluidly couples the compressed gas source with the actuation space.

17. The autoinjector of any of claims 1-15 further including a needle tip fluidly coupled with the puncturing needle opposite to the compressed gas source, the needle tip being configured for axial movement relative to the syringe to pierce the seal whereby axial movement of the needle tip relative to the syringe fluidly couples the actuation space with the puncturing needle.

18. The autoinjector of any of claims 1-17 further including a status indicator indicative of progress of delivery of the injectable drug from the syringe.

19. The autoinjector of claim 18 wherein the status indicator includes a dose indicator.

20. The autoinjector of claim 19 wherein the dose indicator is slidably disposed within a dose indicator window and the dose indicator is tethered to the plunger stopper such that the position of the dose indicator relative to the dose indicator window is indicative of an axial positon of the plunger stopper relative to the syringe.

21. The autoinjector of either of claims 19 and 20 wherein the syringe is configured to provide a series of fractions of the injectable drug.

22. The autoinjector of any of claims 1-21 further including a cap removably coupled to the housing.

23. The autoinjector of any of claims 1-22 the rigid container includes an enlarged neck portion that defines the opening into the interior space, and the non-rigid sealing structure is at least partially disposed within the opening into the opening, and wherein the compressed gas source further includes
a crimping sleeve including a generally cylindrical portion disposed around and crimped below the enlarged neck portion of the rigid container and a generally radially extending portion defining an aperture in alignment with the opening into the interior space, the crimping sleeve being disposed to resist outward movement of the non-rigid sealing structure from the enlarged neck;
a conically shaped rigid structure disposed to exert a sealing force against the non-rigid sealing structure; and
a compressed gas disposed within the interior space of the rigid container.

24. The autoinjector of claim 23 wherein the conically shaped rigid structure includes at least one of the generally radially extending portion of the crimping sleeve being recessed inward, and a conical washer disposed between the generally radially extending portion of the crimping sleeve and the non-rigid sealing structure.

25. The autoinjector of either of claims 23 and 24 further including a pad, the pad being disposed axially along the opening into the interior space and adjacent an outward facing surface of the non-rigid sealing structure.

26. The autoinjector of any of claims 1-25 wherein the puncturing needle connecting the compressed gas source and the actuation space during injection, and connecting a space outside of the syringe and the actuation space at other times.

27. A compact sealed compressed gas source, the compressed gas source comprising:
a rigid container defining an interior space, the rigid container including an enlarged neck portion defining an opening into the interior space;
a non-rigid sealing structure disposed and configured to seal the opening into the interior space, the non-rigid sealing structure being at least partially disposed within the opening into the opening;
a crimping sleeve including a generally cylindrical portion disposed around and crimped below the enlarged neck portion of the rigid container and a generally radially extending portion defining an aperture in alignment with the opening into the interior space, the crimping sleeve being disposed to resist outward movement of the non-rigid sealing structure from the enlarged neck;
a conically shaped rigid structure disposed to exert a sealing force against the non-rigid sealing structure; and
a compressed gas disposed within the interior space of the rigid container.

28. The compact sealed compressed gas source of claim 27 wherein the conically shaped rigid structure includes at least one of the generally radially extending portion of the crimping sleeve being recessed inward, and a conical washer disposed between the generally radially extending portion of the crimping sleeve and the non-rigid sealing structure.

29. The compact sealed compressed gas source of either of claims 27 and 28 further including a pad, the pad being disposed axially along the opening into the interior space and adjacent an outward facing surface of the non-rigid sealing structure.

30. A method of manufacturing the sealed compact gas source of any of claims 26-29 comprising:
inserting the non-rigid sealing structure into the opening into the interior space of the rigid container;
disposing the crimping sleeve around the enlarged neck portion of the rigid container with the conically shaped rigid structure disposed to exert an axially directed sealing force on the non-rigid sealing structure;
crimping the crimping sleeve around the enlarged neck portion by applying a radially inwards deforming force; and
charging the rigid container with a compressed gas.

31. The method of claim 30 when depending from claim 25, further including disposing the conical washer between the generally radially extending portion of the crimping sleeve and the non-rigid sealing structure prior to crimping the crimping sleeve.

32. The method of claim 27 when depending from claims 25 and 26, further including disposing the pad adjacent the outward facing surface of the non-rigid sealing structure.

33. A method of administering an injectable drug comprising fluidly coupling an actuation space of a syringe with a compressed gas source to provide compressed gas to axially translate a plunger stopper within a barrel of the syringe to inject the injectable drug.

34. The method of claim 33 wherein fluidly coupling occurs as a result of an axial translation of a needle safety shield.

35. An injector for use in injection of an injectable drug, the injector comprising:
a syringe including a barrel, a syringe needle fluidly coupled to an interior of the barrel, and a plunger stopper disposed to translate within the barrel, the plunger stopper radially disposed within the barrel, the plunger stopper separating the interior of the barrel into a drug space configured to contain the injectable drug between the plunger stopper and the syringe needle;
a slider axially disposed with the syringe, the slider being rotatably disposed relative to the syringe and substantially axially fixed relative to the syringe;
a member coupled to the plunger stopper, the position of the member being synchronous with the position of the plunger stopper within the barrel; and
a needle safety shield, the needle safety shield being slidably disposed relative to the syringe and the slider whereby an axial force exerted on the needle safety shield slides the needle safety shield relative to the syringe from a shielded position wherein the syringe needle is not axially exposed to an injection position wherein the syringe needle is axially exposed for injection;
wherein the movement of the needle safety shield is at least partially controlled by a pin and track arrangement, one of the pin and the track being formed with the needle safety shield and the other of the pin and the track being formed with the slider whereby movement of the pin within the track controls the position of the needle safety shield relative to the slider, the track including a point-of-no-return, the pin returning the needle safety shield to the shielded position when the axial force is removed when the pin is disposed in the track proximal to the point-of-no-return such that the needle safety shield remains axially movable to the injection position, and the pin moving to and locking the needle safety shield in the shielded position when the axial force is removed when the pin is disposed in the track distal to the point-of-no-return with rotation of the slider;
wherein rotation of the slider is actuated by movement of the member coupled to the plunger stopper, the member rotating the slider positioning the pin distal to the point-of-no-return to axially lock the needle safety shield in the shielded position as the plunger stopper reaches the end of all dose delivery.
